(19) United States
(12) Patent Application Publication
(10) Pub. No.: US 2025/0186481 A1
(43) Pub. Date: Jun. 12, 2025

(54) Multi fiber composition with improved fermentation

(71) Applicant: N.V. Nutricia, Zoetermeer (NL)

(72) Inventors: Sebastian Tims, Utrecht (NL); Lotte Hendrika Johanna Dopheide, Utrecht (NL)

(73) Assignee: N.V. Nutricia, Zoetermeer (NL)

(21) Appl. No.: 19/054,935

(22) Filed: Feb. 17, 2025

Related U.S. Application Data
(63) Continuation of application No. PCT/EP2023/074930, filed on Sep. 11, 2023.

(30) Foreign Application Priority Data
Sep. 9, 2022 (EP) 22194918.3

(51) Int. Cl. A61K 31/717; A23L 33/00; A61K 31/716; A61K 31/718; A61K 31/732; A61K 36/899; A61P 1/14

(52) U.S. Cl. CPC A61K 31/717; A23L 33/40; A61K 31/716; A61K 31/718; A61K 31/732; A61K 36/899; A61P 1/14

(57) ABSTRACT

The invention pertains to a fiber mixture comprising arabinoxylan, beta-glucan, pectin, and resistant starch, nutritional compositions comprising the fiber mixture and uses thereof. The fiber mixture comprising is beneficially used in improving intestinal health and/or preventing impaired intestinal health.

### FIELD OF THE INVENTION

The present invention is in the field of mixtures of dietary fibers and nutritional compositions comprising such mixtures of dietary fibers that have a beneficial fermentation profile and are of particular use in the treatment of patient with coeliac disease.

### BACKGROUND

Dietary fibers are an important part of our diet and are generally seen as essential for health. In adult nutrition the dietary fibers are mainly derived from plant sources and consist of a mixture of soluble and insoluble fiber, a mixture of indigestible poly- and oligosaccharides, and fermentable and non-fermentable fibers. EP 0 756 828 describes a fiber mix with a composition representing the dietary fiber in a typical adult Western diet. Many fibers originating from cereals and grains contain gluten that are capable of stimulating an allergic reaction in patients with Celiac disease.

Celiac disease (CD) is a T-cell mediated enteropathy triggered by the consumption of prolamins from grains such as wheat, barley and rye. Clinical symptoms of CD include fatigue, diarrhoea, abdominal distension, weight loss, anaemia and neurological disorders. CD has been associated with increased rates of intestinal malignancy, such as 10-fold increased risk of intestinal cancer, a 3- to 6-fold increase in the risk of non-Hodgkin lymphoma and a 28-fold increased risk of intestinal T-cell lymphoma as well as increased rates of osteoporosis, and autoimmune disorders such as diabetes.

The only current treatment for CD is lifelong avoidance of dietary gluten, which consists of a family of similar proteins found in wheat (gliadins, glutenins), rye (secalins), barley (hordeins), and oats (avenins). However, such diets are costly and associated with low fiber and high sugar intakes which in themselves are health risks. In addition to suffers from coeliac disease, people who are gluten-intolerant or gluten sensitive are sometimes recommended or prescribed to follow a gluten-free diet. These may include people with Crohn's disease, ulcerative colitis, irritable bowel syndrome, dermatitis herpetiformis, or autism. For some non-coeliac individuals, following a gluten-free diet may be a lifestyle choice.

WO2015/194967 A1 describes a polymer component extracted from lineseed that is suitable for use in baked food products and may be used by subjects with CD. WO 2019/153046 describes compositions to improve gastrointestinal health in a variety of gastrointestinal diseases using plant-based polyphenols and one or more of sweeteners, flavouring agents and a fiber source.

In particular patients on a gluten free diet are at risk to have insufficient fiber intake. Many publications in the art show microbial dysbiosis in patients with Coeliac disease and suggest that the sensitivity in the intestines may be caused by this effect on the microbiome.

When a person with coeliac disease becomes ill the patients has a high risk for malnutrition due to loss of appetite. It appears that medical foods with added fibers are sometimes not well tolerated due to bloating caused by gas production in the large intestines. Therefore, there is a need in the art for an improved fiber composition that can be used in food that both result a high short chain fatty acid production while having a low gas production and can safely be used by patients with Coeliac disease.

### SUMMARY OF THE INVENTION

The present invention overcomes the above problems and provides for a fiber composition having a beneficial short chain fatty acid to gas production ratio. The fiber composition is suitable for patients with Coeliac disease.

The inventors performed faecal fermentation experiments and discovered that a new and optimal fiber composition provides for an unexpected beneficial fermentation profile while maintaining a low gas production and being free from gluten. The fiber mix according to the invention has a short chain fatty acid production to gas production of at least about 100 μmol/ml and at the same time provides for at least 4 mmol short chain fatty acid production/g fiber, wherein the SCFAs assessed are acetic, propionic, n-butyric acid. Such ratio is surprisingly low compared to the effect of the individual fibers as is shown in table 2 and FIG. 1 of example 1. The new fiber mix with 4 fibers resulted in a higher amount of short chain fatty acids produced, in particular in production of a higher amount of acetate. An increased level of acetate in the colon is known to be beneficial for gut health.

The present invention thus provides a new fiber mix comprising arabinoxylan, beta-glucan, pectin, and resistant starch. Further provided is a fiber mixture, or a nutritional composition comprising a fiber mixture, that comprises arabinoxylan, beta-glucan, pectin, and resistant starch. Also provided herein is a fiber mixture, or a nutritional composition comprising a fiber mixture, that comprises arabinoxylan, beta-glucan, pectin, and resistant starch, for use in the treatment of coeliac disease and/or the prevention of the occurrence of symptoms of coeliac disease.

The invention provides for a nutritional composition (powder or liquid) comprising between 0.4-5 g fiber mix per 100 kcal comprising arabinoxylan, beta-glucan, pectin and resistant starch and is gluten free.

### LIST OF FIGURES

The present invention will be discussed in more detail below, with reference to the attached figures.

FIG. 1A. The amounts of acetate, propionate and butyrate produced were determined after 72 hours of faecal slurry fermentation. Mix=mix of the four fibers; AX=arabinoxylan; BG=Beta-glucan; RS=resistant starch. Fiber content in all 5 groups is equal.

FIG. 1B. The cumulative short chain fatty acid (SCFA) to gas production ratio (μmol/ml) was determined after 72 h incubation of faecal samples with arabinoxylan, beta-glucan, pectin, resistant starch and a mixture of these 4 fibers. Mix 1 is the Mix of four fibers AX=arabinoxylan; BG=Beta-glucan; pectin and RS=resistant starch. The fiber content in all 5 samples is equal.

FIG. 1C. The cumulative SCFA to gas ratio of single fiber oat beta-glucan, either being gluten free (first bar) or not gluten free (second bar) was determined after 72 hour of fermentation. Both conditions were standardized for fiber content, from the same supplier and the same nutritional composition. Glutens were enzymatically removed in the beta-glucan in order to be gluten free <20 ppm.

### LIST OF PREFERRED EMBODIMENTS

1. A fiber mixture comprising arabinoxylan, beta-glucan, pectin, and resistant starch.
2. Fiber mixture according to embodiment 1 wherein the mixture is gluten free.
3. Fiber mixture according to embodiments 1 and 2 wherein the arabinoxylan is selected from rice, wheat, psyllium or corn bran arabinoxylan, preferably corn bran arabinoxylan.
4. Fiber mixture according to any of the previous embodiments wherein one or more of the fibers are hydrolysed.
5. Fiber mixture according to any of the previous embodiments wherein pectin is low viscosity pectin having a degree of polymerisation of less than 6000 or a molecular weight of less than 150 kDa.
6. Fiber mixture according to any of the previous embodiments, wherein the mixture comprises at least 75 wt % of fibers selected from the group consisting of arabinoxylan, beta-glucan, pectin, and resistant starch.
7. Fiber mixture according to any of the previous embodiments, wherein the mixture comprises at least 80 wt %, preferably 85 wt % of fibers selected from the group consisting of arabinoxylan, beta-glucan, pectin, and resistant starch.
8. Fiber mixture according to any of the previous embodiment further comprising inulin, fructopolysaccharides, fructooligosaccharides or mixtures thereof.
9. Fiber mixture according to any of the previous embodiments, wherein the mixture comprises between 40-60%, preferably 49% arabinoxylan.
10. Fiber mixture according to any of the previous embodiments, wherein the mixture comprises between 10-30, preferably 21% beta-glucan.
11. Fiber mixture according to any of the previous embodiments, wherein the mixture comprises between 10-30, preferably 18% pectin.
12. Fiber mixture according to any of the previous embodiments, wherein the mixture comprises between 5-25, preferably 12% resistant starch based on total fiber content.
13. Fiber mixture according to any of the previous embodiments wherein the mixture comprises arabinoxylan, beta-glucan, pectin, and resistant starch in a weight ratio of 4-6:1-3:1-3:0.5-2.5, preferably 4.5-5.5:1.5-2.5:1.5-2.5:1-2, more preferably about 5:2:2:1 wherein a 10% margin is included around the individual values.
14. Fiber mixture according to any of the previous embodiments wherein the beta-glucan is oat or barley-derived beta-glucan, preferably oat-derived beta-glucan.
15. Fiber mixture according to any of the previous embodiment wherein the resistant starch is selected from high amylose corn starch, cassava starch or potato starch, preferably high amylose corn starch.
16. A nutritional composition comprising between 0.4 to 5 g per 100 kcal of the fiber mixture according to embodiments 1-15.
17. Nutritional composition according to embodiment 16 wherein the nutritional composition is essentially gluten free.
18. Nutritional composition according to embodiments 16 and 17 wherein the composition is essentially lactose free.
19. A fiber mixture according to embodiments 1 to 15 and a composition according to embodiments 16-18 for use as a medicament.
20. Fiber mixture comprising arabinoxylan, beta-glucan, pectin, and resistant starch, for use in improving intestinal health and/or preventing impaired intestinal health in a subject in need thereof.
21. Fiber mixture for use according to embodiment 20 wherein the subject is a subject with coeliac disease.
22. Fiber mixture for use according to embodiments 20 and 21 wherein the fiber mixture is gluten free and optionally lactose free.
23. Fiber mixture for use according to embodiments 20 to 22 wherein improving intestinal health and/or preventing impaired intestinal health involves 1) an intestinal short chain fatty acid production to intestinal gas production ratio of at least 100 μmol/ml, preferably at least 500 μmol/ml and 2) an intestinal short chain fatty acid production of at least 4 mmol/g fiber mixture.
24. Fiber mixture for use according to embodiments 20 to 23 wherein the fiber mixture comprises arabinoxylan, beta-glucan, pectin, and resistant starch in a weight ratio of 4-6:1-3:1-3:1, more preferably 5:2:2:1.
25. Fiber mixture for use according to embodiments 20 to 24 wherein the fiber mixture is comprised in a nutritional composition.
26. Fiber mixture for use according to embodiments 20 to 25 wherein the mixture comprises arabinoxylan, beta-glucan, pectin, and resistant starch in a weight ratio of 4-6:1-3:1-3:1.

### DETAILED DESCRIPTION OF THE INVENTION

The invention thus pertains to a fiber mixture and compositions comprising the fiber mixture specifically dedicated to being gluten-free and having an optimal fiber composition resulting in surprisingly high short chain fatty acid production while at the same time providing a low gas production thereby preventing impaired intestinal health and/or preventing gluten induced gastrointestinal symptoms.

The present invention thus concerns a fiber mixture comprising arabinoxylan, beta-glucan, pectin, and resistant starch.

The invention also pertains to a nutritional composition comprising a fiber mixture that comprises arabinoxylan, beta-glucan, pectin, and resistant starch.

The invention further concerns a fiber mixture, or a nutritional composition comprising a fiber mixture, that comprises arabinoxylan, beta-glucan, pectin, and resistant starch, for use in improving intestinal health and/or preventing impaired intestinal health and/or treatment of coeliac disease and/or the prevention of the occurrence of symptoms of coeliac disease.

This aspect can also be worded as the use of arabinoxylan, beta-glucan, pectin, and resistant starch in the preparation of a fiber mixture or a nutritional composition for use in improving intestinal health and/or preventing impaired intestinal health and/or the treatment of coeliac disease and/or the prevention of the occurrence of symptoms of coeliac disease.

In other words, the invention pertains to a method for improving intestinal health and/or preventing impaired intestinal health and/or treating coeliac disease and/or the preventing the occurrence of symptoms of coeliac disease in a subject with coeliac disease, by administering a fiber mixture, or a nutritional composition comprising a fiber mixture, that comprises arabinoxylan, beta-glucan, pectin, and resistant starch to the subject.

The present invention also concerns a fiber mixture, or a nutritional composition comprising a fiber mixture, that comprises arabinoxylan, beta-glucan, pectin, and resistant starch, for use in improving intestinal health and/or preventing impaired intestinal health, preferably in a subject with coeliac disease. In an aspect improving intestinal health results from the provision of a beneficial intestinal fermentation profile, the provision of a beneficial ratio of short chain fatty acid production to gas production. Beneficially the fiber mixture is essentially gluten free, preferably free from gluten. The beneficial short chain fatty acid to total gas production and high production of short chain fatty acids per gram of fiber mixture aid in improving intestinal health and/or preventing impaired intestinal health by preventing and/or treating diarrhoea, fatigue, weight loss, bloating and gas, abdominal pain, nausea and vomiting, constipation in a subject with impaired intestinal health, preferably in a subject with coeliac disease.

This aspect can also be worded as the use of arabinoxylan, beta-glucan, pectin, and resistant starch in the preparation of a fiber mixture or a nutritional composition comprising a fiber mixture for use in improving intestinal health and/or preventing impaired intestinal health in a human subject, preferably in a human subject with coeliac disease.

In other words, the invention concerns a method for improving intestinal health and/or preventing impaired intestinal health in a human subject, preferably in a human subject with coeliac disease, by administering a fiber mixture, or a nutritional composition comprising a fiber mixture, that comprises arabinoxylan, beta-glucan, pectin, and resistant starch to the human subject.

The present invention also concerns a fiber mixture, or a nutritional composition comprising a fiber mixture, that comprises arabinoxylan, beta-glucan, pectin, and resistant starch, for use in improving the ratio of colonic short chain fatty acid production to gas production in a human subject, preferably in a human subject with coeliac disease. The fiber mixture according to the invention is a gluten-free fiber mixture.

### Definitions

“Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults.

The term “degree of hydrolysis” refers to the extent to which peptide bonds are broken by a hydrolysis method.

The term “partially hydrolyzed” means having a degree of hydrolysis which is greater than 0 percent but less than about 50 percent.

According to the present invention the term ‘fiber’ or ‘dietary fiber’ means the edible part of plants or analogous carbohydrates that are resistant to digestion and absorption in the human small intestine with complete, partial or no fermentation in the large intestine.

The term “SCFA” as used herein means short chain fatty acid(s) that are produced in the intestinal; tract as an end-product of bacterial fermentation and refers to aliphatic carboxylic acids with a hydrocarbon chain with a hydrocarbon chain with less than six carbon atoms. In a preferred embodiment the short chain fatty acids according to the invention are unbranched fatty acids with 2 (acetic acid or ethanoic acid), three (propionic acid or propionate), four (butyric acid or n-butanoic acid) carbon atoms.

The term “supplement” or “dietary supplement” refers to a nutritional product that provides nutrients to an individual that may otherwise not conveniently be consumed in sufficient quantities by said individual and may be used to complement the nutrition of an individual. It may be in the form of tablets, capsules, pastilles or a liquid and the like. Supplements typically provide the selected nutrients while not representing a significant portion of the overall nutritional needs of the subject. Typically, they do not represent more than 0.1%, 1%, 5%, 10% of the daily energy need of the subject.

The term gluten-free in the current specification refers to products with less than 20 ppm gluten, which is in agreement with the definition from Codex Alimentarius Standard 118-1979.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

### Fiber Mixture

In the context of the present invention, “fiber” and “dietary fiber” are used interchangeably. Fibers are non-digestible carbohydrates. Non-digestible carbohydrates are carbohydrates that are resistant to digestion and absorption in the human stomach and small intestine and enter the colon more or less intact. So, carbohydrates like lactose, maltose, sucrose, standard maltodextrin and standard starch are regarded as digestible carbohydrates. Fibers can be soluble or insoluble in water. The term “soluble fiber” as used herein, when having reference to a non-digestible carbohydrate, is known by the skilled person e.g., according to the method described in the AOAC Official method 991.43 Chapter 32, p7 published in the OAOC Official Methods of Analysis Supplement March 1995. For this patent application soluble fibers sources that comprise more than 60% soluble fibers based on the total fiber content are counted as 100% soluble fiber

Fibers can be fermentable in the colon, or non-fermentable. The term “fermentable” refers to the capability to undergo (anaerobic) breakdown by micro-organisms in the lower part of the gastro-intestinal tract, e.g., colon, to smaller molecules, in particular short chain fatty acids and lactate. The fermentability may be determined by the method described in Am. J. Clin. Nutr. 53, 1418-1424 (1991). Fibers can be as short as a dimer of 2 monomeric carbohydrate moieties but can also have an average degree of polymerization well above 1500. Fibers with a degree of polymerization 2-10 are considered oligomers, whereas fibers with a degree of polymerization above 10 are considered polymers.

Fibers can be hydrolysed into shorter fibers either by enzymatically digestion or chemical treatment. The resulting hydrolysed fibers can have better food technological properties but on the other hand also result in a higher osmolarity depending on the other ingredients of a nutritional composition. In particular high energy or high protein compositions may suffer from viscosity issues and hydrolysed fibers may be necessary to overcome that problem.

The present fiber mixture comprises arabinoxylan, beta-glucan, pectin, and resistant starch. Preferably the mixture comprises arabinoxylan, beta-glucan, pectin, and resistant starch in a weight ratio of 4-6:1-3:1-3:0.5-2.5, more preferably 4.5-5.5:1.5-2.5:1.5-2.5:1-2. In a particular embodiment the mixture comprises arabinoxylan, beta-glucan, pectin, and resistant starch in a weight ratio of about 4-6:1-3:1-3:1, more preferably about 5:2: 2:1.

This ratio of these four fibers ensures an optimal balance and/or interaction between the different types of fibers and their specific beneficial effect, such as a beneficial colonic fermentation profile and a beneficial ratio in colonic short chain fatty acid production to gas production.

### Beta-Glucan

Beta-glucan are polysaccharides of D-glucose monomers linked by beta-glycosidic bonds in various forms such as (1,3)-beta-glucan, (1,4)-beta-glucan, (1,6)-beta-glucan, (1,3;1,6)-beta-glucan and (1,3;1,4)-beta-glucan. The designations (1,3), (1,4) and (1,6) refer to the type of bond found in the beta-glucan and designate the carbon atoms in the D-glucose monomers between which the beta-glycosidic bond is formed. Some, but not all, beta-glucans are water-soluble fibers. Oats are a particularly good source of (1,3;1,4)-beta-glucans, which are generally water soluble, at least at sizes below 2,000,000 Daltons.

Beta-glucans come from the cell walls of bacteria, fungi, yeasts, and some plants and are associated with health effects such as lowering the risk for heart diseases. Beta-glucans are associated with a reduction in both total and low-density lipoprotein (LDL) serum cholesterol and obesity via the satiety effect. Besides, beta-glucans beneficially regulates glycaemic response and has a prebiotic effect which may lower the risk of intestinal diseases. They might also stimulate the immune system by increasing chemicals that prevent infections. The US FDA allows products containing at least 750 mg of beta-glucans to have a health claim stating that they might reduce heart disease risk.

According to the present invention the composition preferably comprises oat or barley-derived beta-glucan, preferably oat and not a microbial source of beta-glucan. Even more preferably the composition according to the present invention comprises a beta-glucan derived from oat.

Oats are not generally considered to contain gluten, and although oats comprise a protein from the prolamin family, avenin, this does generally not present a health problem for most people who are sensitive to gluten. Consequently, oat products are often considered to be suitable for coeliac sufferers, provided there is no contamination from other grains which do contain gluten, such as wheat, barley and rye, and oats and oat-derived products are often recommended for those on a gluten-free diet.

In a preferred embodiment, the fiber mixture comprises beta-glucan. Beta-glucan is preferably present in the fiber mixture in a range of 10-30 wt percent, preferably 15-25 wt percent based on total weight of the fibers. In some embodiments 21 wt percent of resistant starch is present in the fiber mixture.

### Arabinoxylan

Arabinoxylans are hemicellulose dietary fibers abundant in the bran of many cereal grains including wheat, oat, rice, psyllium and corn. They pass undigested through the human intestine until they reach the colon where they provide substrates for bacterial fermentation, generating beneficial short-chain fatty acids including acetate, butyrate and propionate. As viscous fibers, arabinoxylans help suppress appetite, delay gastric emptying and slow post-prandial glucose and insulin responses as well as promote laxation. Their beneficial effects on post-prandial glucose responses have been recognised by the European Food Safety Authority, and the European Union has authorised a health claim that ‘consumption of arabinoxylan as part of a meal contributes to a reduction of the blood glucose rise after that meal’.

According to the present invention the arabinoxylan is preferably from oat, wheat, psyllium, corn or rice. Arabinoxylan derived from oat, psyllium, wheat, corn or rice as used in the context of the invention is beneficially gluten free. Most preferably the fiber mixture according to the invention comprises exclusively arabinoxylan from corn.

In a preferred embodiment, the fiber mixture comprises arabinoxylan. Arabinoxylan is preferably present in the fiber mixture in a range of 40-60 wt percent, preferably 45-55 wt percent based on total weight of the fibers. In some embodiments 49 wt percent of arabinoxylan is present in the fiber mixture.

### Resistant Starch

Resistant starch (RS) generally consists of any type of starch that escapes digestion and intestinal absorption and can be formed during processing of starchy foods. Resistant starch may also be obtained from raw foods, such as potatoes or green bananas. Resistant starch is normally insoluble and the fermentability varies with the source. Preferably, resistant starch is present in the fiber mixture of the invention.

RS can be determined according to the method described by McCleady and Monaghan (2002) J AOAC Int 85, 665-675. RS has been categorized into four types:

RS1 refers to physically inaccessible or undigestible resistant starch, such as that found in seeds or legumes and unprocessed whole grains. RS2 refers to resistant starch that is inaccessible to enzymes due to starch conformation, as in green bananas and high amylose corn starch. RS3 refers to resistant starch that is formed when starch-containing foods are cooked and cooled, such as pasta. RS3 occurs due to retrogradation, which refers to the collective processes of dissolved starch becoming less soluble after being heated and dissolved in water and then cooled. RS4 refers in turn to starches that have been chemically modified to resist digestion.

Preferably the resistant starch is retrograded starch and/or starch that is chemically modified to resist digestion, most preferably retrograded starch. A suitable source of resistant starch is Novelose@330 (Ingredion).

Preferably the fiber mixture according to the invention comprises resistant starch from natural origin, preferably an RS1, RS2 or RS3 type starch, most preferably the resistant starch is of type RS3 such as high amylose corn starch, cassava starch or potato starch. Preferably the resistant starch is a retrograded [RS3] resistant high amylose starch, preferably from corn.

Resistant starch together with the other fibers of the fiber mixture according to the invention advantageously results in the formation of a beneficial intestinal fermentation profile and the provision of a beneficial ratio of short chain fatty acid production to gas production.

In a preferred embodiment, the fiber mixture comprises resistant starch. Resistant starch is preferably present in the fiber mixture in a range of 5-25 wt percent, preferably 10-20 wt percent based on total weight of the fibers. In some embodiments 12 wt percent of resistant starch is present in the fiber mixture.

### Pectin

Pectins, also known as pectic polysaccharides, are rich in galacturonic acid. Several distinct polysaccharides have been identified and characterised within the pectic group. Homogalacturonans are linear chains of a-(1-4)-linked D-galacturonic acid. Pectin fibers are obtainable from fruits and vegetables and are generally rich in galacturonic acid groups. Pectins are used in food as stabilizer or for the gelling properties of pectin.

In nature, around 80 percent of carboxyl groups of galacturonic acid are esterified with methanol. This proportion is decreased to a varying degree during pectin extraction. Pectins are classified as high-vs. low-methoxy pectins (short HM-pectins vs. LM-pectins), with more or less than 50 percent of all the galacturonic acids esterified respectively. The ratio of esterified to non-esterified galacturonic acid determines the behaviour of pectin in food applications. For the present invention pectin is not used as stabilizer or thickener, on the contrary we wish to keep the viscosity of the product as low as possible.

Pectins can be hydrolyzed in order to decrease the molecular weight. For the present application partially hydrolyzed pectins are preferred in order to prevent undesired increases in viscosity.

Therefore, preferably the pectin according to the present invention is a partially hydrolysed pectin, also called a low viscosity pectin, that will have a limited effect on viscosity but still is a good source of fermentable fiber. In a preferred embodiment the low viscosity pectin has a degree of polymerisation of less than 6000 or a molecular weight of less than 150 kDa such as e.g., Herbapekt LV.

Preferably the pectin used in the fiber mixture is obtained from citrus or an apple source and mildly hydrolysed to prevent increasing the viscosity of the end product.

In a preferred embodiment, the fiber mixture comprises pectin. Pectin is preferably present in the fiber mixture in a range of 10-30 wt percent, preferably 15-25 wt percent, preferably 17-23 wt percent, based on total weight of the fibers. In some embodiments 18 wt percent of pectin is present in the fiber mixture.

### Gluten Free

The term gluten-free in the present invention is intended to describe that compositions strictly exclude gluten, which is a mixture of proteins found in wheat (and all of its species and hybrids, such as spelt, kamut, and triticale), as well as barley, and rye.

Inherently gluten-free diets (GFD) are low in fiber as the main gluten containing food sources are also the high fiber ones, i.e., whole grain vs. refined grain. Mainly whole grains are diminished from the GFD. Concerning the importance of adequate and diverse fibers type intake (they all exert a range of health benefits and stronger stimulate microbial diversity over single fiber consumption), it is of great importance to have gluten free grain derived fiber types in a gluten free fiber mixture. GFD are recommended for coeliac disease (CD) and non-celiac gluten sensitivity, however also many healthy individuals apply to a GFD. Adequate fiber intake results in a range of health benefits from metabolic markers to gut microbiota diversity and immune health.

The fiber mixture and compositions comprising the fiber mixture according to the present invention are specifically dedicated to being gluten-free and having an optimal fiber composition resulting in surprisingly high short chain fatty acid production while at the same time providing a low gas production thereby preventing impaired intestinal health and/or preventing gluten induced gastrointestinal symptoms. The fiber mixture and compositions comprising the fiber mixture according to the present invention are particularly beneficial for subjects adhering to a gluten-free diet, in particular subject with Coeliac disease.

### Lactose

Many subjects with impaired intestinal health and Coeliakie patients are lactose intolerant and these subjects would greatly benefit of a diet low in lactose. The fiber mixture and compositions comprising the fiber mixture according to the present invention are preferably low in lactose or even more preferably are lactose free. Low in lactose in the context of the invention means a composition with less than 1000 ppm lactose. Low in lactose preferably means herein that the lactose content of the fiber mixture or of compositions comprising the fiber mixture according to the present invention is at most 1000 ppm, preferably at most 800 ppm, more preferably at most 600 ppm, most preferably at most 400 ppm and in particular at most 300 ppm.

It is believed that fiber mixtures and compositions comprising the fiber mixture according to the present invention that are low in lactose aid in providing a low intestinal gas production thereby preventing impaired intestinal health and/or preventing diet induced gastrointestinal symptoms. In a further preferred embodiment, the fiber mixture or nutritional compositions comprising the fiber mixture are lactose free.

### Subject

In the context of the present invention, a toddler is a subject between 1 and 3 years, a child a subject between 3 and 12 years, a teenager a subject between 12 and 18 years, an adult a subject older than 18 years. Preferably the subject is older than 1 year, more preferably 3 years, even more preferably 12 years, even more preferably 14 years most preferably 18 years. Preferably the subject is a toddler, child, teenager or adult, more preferably the patient is a child, teenager or adult; even more preferably the patient is a teenager or adult, most preferably the patient is an adult. When referred to a patient or subject, it concerns human patients or subjects.

### Nutritional Composition

In one embodiment the present invention also concerns a nutritional composition comprising the fiber mixture according to the present invention. In one embodiment the present nutritional composition is a liquid. In one embodiment, preferably the present nutritional composition is a ready-to-feed composition. Preferably the composition is administered orally. In one embodiment the present nutritional composition is in a powdered form, which can be reconstituted with water to form a liquid. In the context of the present invention the terms ‘powder’ and ‘dry’ are used interchangeably. The fiber mixture and the nutritional composition comprising the fiber mixture according to the invention are preferably gluten-free.

In one embodiment a nutritional composition according to the invention comprises 0.4-10 g of the present fiber mixture per 100 ml, preferably 0.6-8 g, more preferably 0.8-5 g and even more preferably 1-4 g of the present fiber mixture per 100 ml nutritional composition.

When in powder form, preferably the nutritional composition according to the invention comprises 0.5-45 g of the present fiber mixture per 100 g dry weight, preferably 2-40 g, more preferably 4-25 g and even more preferably 5-15 g of the present fiber mixture per 100 g dry weight of the nutritional composition.

Based on calories, preferably the nutritional composition according to the invention comprises 0.4-50 g of the fiber mixture per 100 kcal, preferably 0.6-25 g of the present fiber mixture per 100 kcal, preferably 0.5-12 g, more preferably 0.6-7.5 g and even more preferably 0.7-5 g of the fiber mixture per 100 kcal, based on the total energy content of the nutritional composition (including the fibers). In some embodiments the nutritional composition according to the invention comprises 0.4-5 g of the fiber mixture per 100 kcal. For the purpose of the present invention the caloric density of the fibers is set at 2 kcal per gram.

In case the composition according to the invention is a complete nutritional composition, the dietary fiber content is preferably 0.4-9 g dietary fibers per 100 kcal, even more preferably 0.6-7 g per 100 kcal and most preferably 0.7-5 g per 100 kcal of the composition. In case the composition according to the invention is a supplement, the dietary fiber content is preferably 2-50 g dietary fibers per 100 kcal, even more preferably between 4-30 g per 100 kcal and most preferably 5-20 g per 100 kcal of the composition. The actual dose depends on the age and weight of the patient. For infants and children up to 12 years the preferred daily dose is between 2 and 15 g and for adults the preferred daily dose is between 7 and 35 g dietary fibers.

In a preferred embodiment a nutritional composition according to the invention comprises between 50 and 350 kcal per 100 ml, and between 0.5 and 5 g dietary fiber per 100 kcal, more preferably between 0.7 and 4.5 g, even more preferably between 0.8 and 4.0 g dietary fiber per 100 kcal of the nutritional composition.

The quantity of fibers promotes the advantageous effects of these fibers in the gastro-intestinal tract yet is suitable for subject with Coeliac disease and does minimize the risk of unwanted side effects such as bloating, abdominal pain, flatulence and/or a feeling of satiety. The amount of fiber can suitably be determined according to McCleary, Anal Bioanal Chem 25 2007, 389:291-308. This method suitably determines total fiber including resistant starch and nondigestible oligosaccharides.

The nutritional composition according to the invention may in an embodiment comprise digestible carbohydrate, lipid and protein. The lipid preferably provides 10-45% of the total calories, the protein preferably provides 5-35% of the total calories and the digestible carbohydrate preferably provides 25-65% of the total calories of the nutritional composition. Preferably a nutritional composition according to the invention comprises lipid providing 25-45% of the total calories, protein providing 6-35% of the total calories and digestible carbohydrate providing 30-50% of the total calories of the nutritional composition. More preferably the present nutritional composition comprises lipid providing 30-45% of the total calories, protein providing 7-10% of the total calories and digestible carbohydrate providing 35-45% of the total calories of the nutritional composition. The energy provided by nutrients is calculated using the Atwater calculation factors 9 kcal per g lipid, 4 kcal per gram protein or gram digestible carbohydrate and zero kcal for the other components.

The nutritional composition preferably comprises 1-25 g lipid per 100 ml, more preferably 2.0-20 g per 100 ml, more preferably 2.5-13 g per 100 ml. Based on dry weight, the nutritional composition preferably comprises 4-45 g lipid per 100 g, more preferably 10-35 g per 100 g, more preferably 15-30 g lipid per 100 g dry weight of the nutritional composition. Based on calories, the nutritional composition preferably comprises 2.0-7.0 g lipid per 100 kcal, more preferably 2.5-6.0 g per 100 kcal, more preferably 3.0-5.0 lipid g per 100 kcal of the nutritional composition.

The lipid preferably provides 20-55%, more preferably 25-50%, more preferably 30-45% of the total calories of the present nutritional composition. The amount of saturated fatty acids is preferably below 25 wt. % based on total lipids more preferably below 15 wt. %. The concentration of monounsaturated fatty acids preferably ranges from 20 to 65% based on weight of total fatty acids. The concentration of polyunsaturated fatty acids preferably ranges from 15 to 60% based on weight of total fatty acids. Preferably the nutritional composition comprises the n-6 polyunsaturated fatty acid linoleic acid (LA)

Preferably the nutritional composition comprises long chain poly-unsaturated fatty acids (LCPUFA). LC-PUFA are defined in the present invention as fatty acids or acyl chains with two or more double bonds and a chain length of 20 or above. Preferably the nutritional composition comprises docosahexaenoic acid (DHA) and/or eicosapentaenoic acid (EPA). DHA and EPA are n-3 LC-PUFA which play a role in preventing intestinal inflammation and intestinal pain, thereby further improving the intestinal health.

The nutritional composition preferably comprises 0.8-18 g protein per 100 ml, more preferably 1.0-18 g per 100 ml, more preferably 2-18 and most preferably 5-18 g per 100 ml nutritional composition. Based on dry weight, the nutritional composition preferably comprises at least 6 gram protein per 100 g, preferably 6-55 g protein per 100 g, more preferably 7-55 g per 100 g, more preferably 8-55 g protein per 100 g dry weight of the nutritional composition.

Based on calories, the nutritional composition preferably comprises 1.2-10.0 g protein per 100 kcal, more preferably 1.5-9.0 g per 100 kcal, more preferably 1.7-8.0 g protein per 100 kcal of the nutritional composition.

Protein is to be taken as the sum of proteins, peptides and free amino acids. Measurement of protein amount can be done according to standard procedures known to the skilled person.

The present nutritional composition in an embodiment comprises casein and/or whey proteins. Preferably the weight ratio casein: whey protein is 0:100 to 90:10, more preferably 20:80 to 90:10, more preferably 40:60 to 80:20. In an alternative embodiment the nutritional composition comprises plant-derived proteins such as but not limited to, soy, pea, potato, rice protein or mixtures thereof.

The nutritional composition preferably comprises 15-65 g digestible carbohydrates per 100 ml, more preferably 18-60 g per 100 ml, more preferably 20-50 g per 100 ml nutritional composition. Based on dry weight, the nutritional composition preferably comprises 30-80 g digestible carbohydrates per 100 g, more preferably 35-70 g per 100 g, more preferably 40-60 g digestible carbohydrates per 100 g dry weight of the nutritional composition.

Based on calories, the nutritional composition preferably comprises 7-20 g digestible carbohydrates per 100 kcal, more preferably 10-18 g per 100 kcal, more preferably 12-15 g digestible carbohydrates per 100 kcal of the nutritional composition and wherein the digestible carbohydrates preferably provides 20-75%, more preferably 25-65%, more preferably 30-55% of the total calories of the present nutritional composition.

Preferably the nutritional composition comprising the fiber mixture according to the invention comprises at least one digestible carbohydrate selected from the group consisting of maltodextrin, digestible starch, saccharose, glucose, and maltose. The nutritional composition comprising the fiber mixture according to the invention is preferably low in lactose, more preferably the composition is essentially lactose-free. Preferably the composition comprises less than 1000 ppm lactose.

The fiber mixture according to the present invention does not exclude that other fibers, or in other words non-digestible carbohydrates, such as human milk oligosaccharides, may be present, provided said other fibers are gluten-free. Thus, in one embodiment, the nutritional composition according to the invention comprises at least 70 wt %, preferably at least 80 wt %, more preferably at least 90 wt %, of the present fiber mixture based on total fibers. In one embodiment, the fibers present in the nutritional composition consist of the fiber mixture according to the present invention.

In terms of individual fibers the nutritional composition according to the invention preferably comprises at least 0.08 g beta-glucan per 100 ml, more preferably at least 0.1 g, even more preferably at least 0.2 g beta-glucan per 100 ml. Based on dry weight, the nutritional composition according to the invention preferably comprises at least 1 g beta-glucan per 100 g, more preferably at least 1.5 g, even more preferably at least 3 g beta-glucan per 100 g. Based on calories, the nutritional composition according to the invention preferably comprises at least 0.12 g beta-glucan per 100 kcal, more preferably at least 0.15 g, even more preferably at least 0.3 g beta-glucan per 100 kcal.

The nutritional composition according to the invention preferably comprises less than 2 g beta-glucan per 100 ml, more preferably less than 1.5 g, even more preferably less than 1.0 g beta-glucan per 100 ml. Based on dry weight, the nutritional composition according to the invention preferably comprises less than 15 g beta-glucan per 100 g, more preferably less than 10 g, even more preferably less than 7.5 g beta-glucan per 100 g. Based on calories, the nutritional composition according to the invention preferably comprises less than 3 g beta-glucan per 100 kcal, more preferably less than 2.5 g, even more preferably less than 1.5 g beta-glucan per 100 kcal.

Furthermore, the nutritional composition according to the invention preferably comprises at least 0.4 g arabinoxylan per 100 ml, more preferably at least 0.8 g, even more preferably at least 1.0 g arabinoxylan per 100 ml. Based on dry weight, the nutritional composition according to the invention preferably comprises at least 2.0 g arabinoxylan per 100 g, more preferably at least 2.5 g, even more preferably at least 3.0 g arabinoxylan per 100 g. Based on calories, the nutritional composition according to the invention preferably comprises at least 0.4 g arabinoxylan per 100 kcal, more preferably at least 0.5 g, even more preferably at least 0.6 g arabinoxylan per 100 kcal.

The nutritional composition according to the invention preferably comprises less than 4 g arabinoxylan per 100 ml, more preferably less than 3 g, even more preferably less than 2.5 g arabinoxylan per 100 ml. Based on dry weight, the nutritional composition according to the invention preferably comprises less than 15 g arabinoxylan per 100 g, more preferably less than 12.5 g, even more preferably less than 10 g arabinoxylan per 100 g. Based on calories, the nutritional composition according to the invention preferably comprises less than 6 g arabinoxylan per 100 kcal, more preferably less than 4.5 g, even more preferably less than 3 arabinoxylan per 100 kcal.

Additionally, the nutritional composition according to the invention preferably comprises at least 0.025 g resistant starch per 100 ml, more preferably at least 0.05 g, even more preferably at least 0.1 g resistant starch per 100 ml. Based on dry weight, the nutritional composition according to the invention preferably comprises at least 0.2 g resistant starch per 100 g, more preferably at least 0.25 g, even more preferably at least 0.3 g resistant starch per 100 g. Based on calories, the nutritional composition according to the invention preferably comprises at least 0.02 g resistant starch per 100 kcal, more preferably at least 0.03 g, even more preferably at least 0.04 g resistant starch per 100 kcal.

The nutritional composition according to the invention preferably comprises less than 1 g resistant starch per 100 ml, more preferably less than 0.75 g, even more preferably less than 0.5 g resistant starch per 100 ml. Based on dry weight, the nutritional composition according to the invention preferably comprises less than 4.0 g resistant starch per 100 g, more preferably less than 3.0 g, even more preferably less than 2.0 g resistant starch per 100 g. Based on calories, the nutritional composition according to the invention preferably comprises less than 0.6 g resistant starch per 100 kcal, more preferably less than 0.45 g, even more preferably less than 0.3 g resistant starch per 100 kcal.

Additionally, the nutritional composition according to the invention preferably comprises at least 0.1 g pectin per 100 ml, more preferably at least 0.25 g, even more preferably at least 0.4 g pectin per 100 ml. Based on dry weight, the nutritional composition according to the invention preferably comprises at least 0.5 g pectin per 100 g, more preferably at least 1.0 g, even more preferably at least 1.5 g pectin per 100 g. Based on calories, the nutritional composition according to the invention preferably comprises at least 0.04 g pectin per 100 kcal, more preferably at least 0.06 g, even more preferably at least 0.08 g pectin per 100 kcal.

The nutritional composition according to the invention preferably comprises less than 1.5 pectin per 100ml, more preferably less than 1.25 g, even more preferably less than 1.0 g pectin per 100 ml. Based on dry weight, the nutritional composition according to the invention preferably comprises less than 5.0 g pectin per 100 g, more preferably less than 4.0 g, even more preferably less than 3.0 g pectin per 100 g. Based on calories, the nutritional composition according to the invention preferably comprises less than 0.8 g pectin per 100 kcal, more preferably less than 0.6 g, even more preferably less than 0.4 g pectin per 100 kcal.

Preferably the nutritional composition further comprises vitamins, minerals and trace elements and other micronutrients in recommended daily amounts as known in the art and according to international guidelines.

The osmolarity of the present nutritional composition is preferably between 150 and 900 mOsmol/l, more preferably 180 to 800 mOsmol/l. This osmolarity advantageously reduced gastro-intestinal stress, results in an optimal balance between water and nutrient uptake. Fibers intent to increase the osmolarity significantly. The fiber composition according to the invention is also selected to prevent a increased osmolarity that could potentially further increase gut irritation in patients suffering from Coeliac disease.

### Supplement

The fiber mixture according to the invention may in a preferred aspect be used as a nutritional supplement, i.e., as an additive to a diet. The supplement, preferably for enteral application, may be a solid or liquid galenical formulation. Examples of solid galenical formulations are tablets, capsules (e.g. hard or soft shell gelatine capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as flavouring agents, preservatives, stabilizers, emulsifying agents, buffers and the like may be added in accordance with accepted practices of nutritional and pharmaceutical compounding.

### Application

In one embodiment, the present fiber mixture, or nutritional composition comprising the fiber mixture, is for adult subjects and for pediatric patients. In the context of the present invention, pediatric patients relate to toddlers and young children and adolescent human subjects from 1 up to and including 12 years of age who are under medical supervision for a disorder. More particular, the present fiber mixture, or nutritional composition comprising the fiber mixture, is for adult and pediatric patients requiring nutritional support. In this context the adult or pediatric patient having a disorder and/or requiring nutritional support has Coeliac disease.

A preferred daily dosage of the fiber mixture according to the present invention is about 0.9-100 g per day, preferably about 1.2-50 g, more preferably 3-35 g, even more preferably 6-30 g fiber mixture per day.

When the nutritional composition according to the invention is in a liquid form, or reconstituted into its liquid form, the preferred volume of the nutritional composition that is administered on a daily basis is in the range of about 100 to 2500 ml, more preferably about 200 to 2000 ml per day, even more preferably about 300 to 500 ml per day.

### EXAMPLES

### Example 1

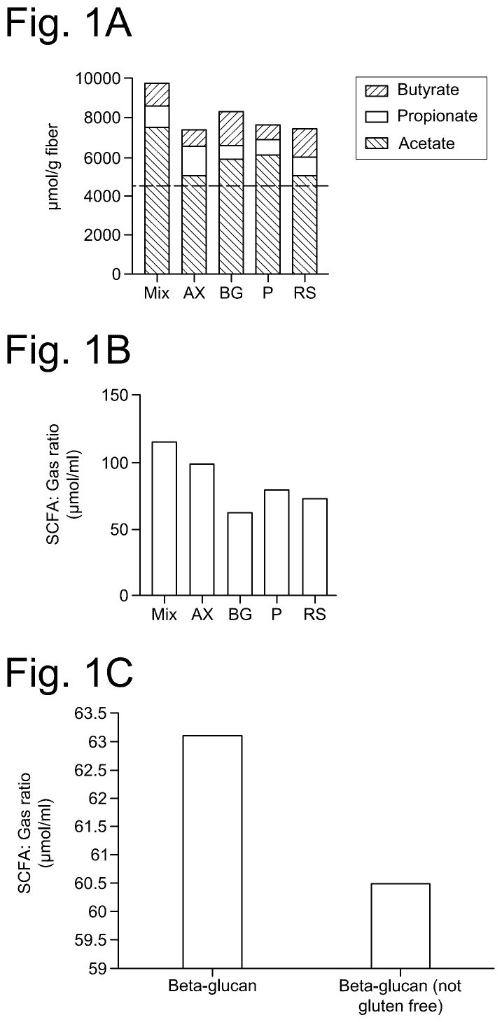

To study the fermentation of different fibers a faecal slurry fermentation experiment was performed. Fresh faecal samples were collected from 5 Caucasian adults, 2 male and 3 female, aged 23-48 years. The volunteers were without gastrointestinal problems and did not use antibiotics in the 4 months preceding the donation. Faecal samples were pooled, homogenized, divided in smaller aliquots, and mixed with glycerol (10%) in an anaerobic cabinet. Subsequent aliquot storage was at −80° C. The negative control was a blank experiment performed with faecal samples without any substrate at pH 6.3. Faecal samples fed with glucose were used as positive control. Two fermentation runs were performed: one with each fiber as a single ingredient (arabinoxylan, beta-glucan, pectin or resistant starch) and one run with a mixture of the fibers with 49% arabinoxylan, 21% beta-glucan 18% pectin and 12% resistant starch based on total fiber content (Table 1). Except for the oat beta-glucan in FIG. 1C, the fibers were all gluten free. In each run the negative and positive controls were included and all condition started at pH 6.3. Fibers (DP≥3) and glucose were added at a concentration of 200 mg per 6 ml of faeces suspension.

TABLE 1
Fibers included in the faecal fermentation.

| | | % fiber |
|---|---|---|
| | | content |
| Substrate | Commercial source | (+/−10%) |
| Arabinoxylan | Corn arabinoxylan (Pure Fiber) | 79 |
| Beta-glucan | PromOat Gluten Free (Lantmännen) | 31 |
| Pectin | SF 50 A-LV (Herbafood) | 81 |
| Resistant starch | Novelose 330 (Ingredion) | 41 |
| Fiber mix of | mixture with 49% arabinoxylan, 21% | 65 |
| arabinoxylan, beta- | beta-glucan 18% pectin and 12% | |
| glucan, pectin, | resistant starch made of the above | |
| and resistant starch | individual commercial sources. | |

* The percentage of fiber is the part of the substrate that contains carbohydrate polymers with a DP equal or higher than 3.

Before the experiment the faecal pool was defrosted in a water bath for 20 minutes at 37° C. The faecal pool was put thereafter in the anaerobic cabinet. Faeces was mixed with the fermentation medium as 1:5 in a falcon tube. Samples of this faecal suspension were taken at t=0 and 6 ml of this suspension was added to a sterile falcon tube together with the substrate of interest and mixed thoroughly. Next, 6 ml of the faeces/substrate suspension was put in a dialysis tube and air was removed in the empty space. The dialysis tube was put in a 100 ml Scott bottle filled with 100 ml dialysis medium. The Scott bottles were closed and incubated at 37° C. Samples of the dialysis medium (dialysate) and faecal suspension (lumen) were taken at t=24, t=48, and t=72 hours for determination of short chain fatty acids (SCFA), ammonia, D-and L-lactate, pH and gas volume.

Fermentation medium (Mc Bain and MacFarlane) contains buffered peptone water 3.0 g/l, Yeast Extract 2.5 g/l, Tryptone 3.0 g/l, L-Cysteine-HCl 0.4 g/l, Bile salts 0.05 g/l, K₂HPO₄.3H₂O 2.6 g/l, NaHCO₃ 0.2 g/l, NaCl 4.5 g/l, MgSO₄.7H₂O 0.5 g/l, CaCl₂. 2H₂O 0.3 g/l, FeSO₄.7H₂O 0.005 g/l. Ingredients were added one by one in 800 ml water, pH was adjusted to 6.3±0.1 with K₂HPO₄ or NaHCO₃ and volume was filled up to 1 litre. Medium was sterilized for 15 minutes at 121° C. and put in the anaerobic cabinet at least 16 hours before use.

Dialysis medium contains K₂HPO₄.3H₂O 2.6 g/l, NaHCO₃ 0.2 g/l, NaCl 4.5 g/l, MgSO₄.7H₂O 0.5 g/l, CaCl₂. 2H₂O 0.3 g/l, FeSO₄.7H₂O 0.005 g/l. pH was adjusted to 6.3±0.1 with K₂HPO₄ or NaHCO₃. Medium was not sterilized because of the formation of sediment. The medium was put in the anaerobic cabinet at least 16 hours before use.

The pH was measured by immersing a 423 pH-electrode (Mettler Toledo, Columbus, OH, USA), connected to a Handy-lab PH meter (Schott Glas, Mainz, Germany), directly in a sample.

Gas volume was determined at 3 different time points (24, 48 and 72 hours) with a unit to measure pressure and volume. The bottles were shaken thoroughly before measuring.

The SCFA acetic acid, propionic acid, n-butyric and were quantitatively determined at 3 different time points (24, 48 and 72 hours) using a Shimadzu-GC2025 gas chromatograph with a flame ionization detector. Hydrogen was used as mobile phase. The levels of SCFA were determined using 2-ethylbutyric acid as an internal standard. From the peak area a calibration curve was constructed and the concentration in the samples was calculated.

It was observed that the fiber mixture comprising arabinoxylan, beta-glucan, pectin and resistant starch resulted in low amounts of gas production in terms of volume at all 3 time points. At 24 hours the fiber mixture resulted in a mean gas volume production of 47 ml per gram fiber which was surprisingly lower than the amounts of gas produced with the individual fibers. At all 3 time points the total amount of short chain fatty acids produced per gram of fiber was higher for the mixture of fibers than for the individual fibers (Table 2). Together the mixture of fibers provided at all time point for a beneficially and significantly increased ratio of the total amount of SCFA over the total gas production.

TABLE 2
Cumulative gas production, total SCFA production, and the ratio of
total SCFA to gas during the 72-hour faecal slurry fermentation.

| Time | | Gas volume | | Total SCFA | | SCFA/Gas | |
|---|---|---|---|---|---|---|---|
| point | | (ml/g fiber) | | (μmol/g fiber) | | (μmol/ml) | |
| (hours) | Fibers/mix | Mean | SD | Mean | SD | Mean | SD |
| 24 | Blanc | 18.50 | 0.71 | 2145.0 | 162.6 | 116.2 | 13.2 |
| | Arabinoxylan | 54.00 | 7.07 | 3470.0 | 113.1 | 65.0 | 10.6 |
| | Beta-glucan | 66.75 | 17.32 | 5082.5 | 1269.3 | 76.2 | 0.8 |
| | Pectin | 60.75 | 7.42 | 3925.0 | 77.8 | 65.0 | 6.7 |
| | Resistant | 47.75 | 1.06 | 3485.0 | 473.8 | 72.9 | 8.3 |
| | starch | | | | | | |
| | Mix | 47.00 | 9.90 | 5397.1 | 141.4 | 117.8 | 27.8 |
| 48 | Blanc | 23.00 | 1.41 | 3970.0 | 1032.4 | 171.6 | 34.3 |
| | Arabinoxylan | 71.00 | 11.31 | 6305.0 | 261.6 | 89.6 | 10.6 |
| | Beta-glucan | 104.25 | 13.79 | 6917.5 | 1219.8 | 66.2 | 2.9 |
| | Pectin | 90.75 | 0.35 | 6567.5 | 215.7 | 72.4 | 2.1 |
| | Resistant | 70.25 | 4.60 | 5502.5 | 668.2 | 78.2 | 4.4 |
| | starch | | | | | | |
| | Mix | 74.50 | 9.90 | 7239.6 | 187.4 | 98.2 | 15.6 |
| 72 | Blanc | 26.50 | 2.12 | 4467.5 | 1007.6 | 167.6 | 24.6 |
| | Arabinoxylan | 75.50 | 12.02 | 7412.5 | 399.5 | 99.0 | 10.5 |
| | Beta-glucan | 131.75 | 10.25 | 8332.5 | 1156.1 | 63.1 | 3.9 |
| | Pectin | 96.25 | 0.35 | 7655.0 | 56.6 | 79.5 | 0.9 |
| | Resistant | 100.25 | 4.60 | 7410.0 | 770.7 | 73.8 | 4.3 |
| | starch | | | | | | |
| | Mix | 85.50 | 11.31 | 9782.1 | 14.1 | 115.4 | 15.1 |
| 24 | Beta-glucan | 61.50 | 24.75 | 3682.5 | 392.4 | 63.8 | 19.3 |
| | (not gluten | | | | | | |
| | free) | | | | | | |
| 48 | Beta-glucan | 90.50 | 47.38 | 5950.0 | 742.5 | 73.7 | 30.4 |
| | (not gluten | | | | | | |
| | free) | | | | | | |
| 72 | Beta-glucan | 120.50 | 47.38 | 6870.0 | 749.5 | 60.5 | 17.6 |
| | (not gluten | | | | | | |
| | free) | | | | | | |

The amount of acetate, propionate and butyrate produced after 72 hours of faecal slurry fermentation were further determined. It was observed that the fiber mixture shows higher SCFA production in μmol per gram fiber as compared to the individual fibers (FIG. 1A).

Although the total fiber content in all experimental groups was the same, the gluten-free fiber mixture of arabinoxylan, beta-glucan, pectin and resistant starch according to the invention had a surprisingly improved SCFA: gas ratio compared to the individual fibers (FIG. 1B).

Further, gluten free beta-glucan, as used in above experiment, was compared to non-gluten free beta-glucan (from the same supplier). The oat-beta glucan was enzymatically treated to achieve <20 ppm gluten levels in the gluten free version.

Surprisingly gluten free oat beta-glucan was more effective in, i.e., led to a higher SCFA to gas ratio in μmol/ml, SCFA production as was the non-gluten free version (FIG. 1C). This experiment shows that the beta-glucan suitable for Coeliac disease patients is even the more preferred source of fiber individually or to implement in a fiber mixture for improving the SCFA/gas ratio.

### Example 2

### Nutritional Product Suitable for Coeliac Patients >36 Months of Age

A liquid nutritionally complete food composition was prepared to be preferably consumed orally, either as sole source of nutrition or supplement by subjects >36 months of age. Per 100 kcal, the composition comprises of 8.5-15-gram digestible carbohydrates and 0.8-2-gram fibers in the composition as shown in example 1. Proteins either derived from plant-based or non-plant-based sources, contributing to 2.5-9 grams per 100 kcal. Fat contributing to 2-5 grams per 100 kcal. Micronutrients were included in quantities as known in the art.

### Example 3

### Nutritional Product Suitable for Coeliac Patients >36 Months

A nutritionally complete medical liquid food composition was prepared to be orally and preferably enterally consumed, either as sole source of nutrition or supplement by subjects >36 months of age. The product is gluten free (<20 ppm) and low in lactose (<1000 ppm). Per 100 mL, this composition was prepared by mixing 1-3 gram fiber in the following combination: 49% arabinoxylan, 21% beta-glucan, 18% pectin and 12% resistant starch. 10-35 En % protein, either intact or isolates and 26-65 En % carbohydrates with <20% sugars. Fats contributed to 11-45 En %, preferably predominantly unsaturated fatty acids. A vitamin blend comprising at least vitamin D 0.5-3 microgram and a mineral mix comprising at least calcium at a dose of 2-150 mg was included. Optionally flavors, colorants etc. were added.

### Example 4

Nutritional Product Suitable for coeliac Patients >36 Months

A liquid nutritionally complete food composition was prepared to be preferably consumed orally, either to be consumed as sole source of nutrition or as a supplement by subjects >36 months of age. The product is gluten free (<20 ppm) and low in lactose (<1000 ppm). Per 100 mL, the composition comprises of 1-4 grams fiber per 100 mL in the combination as shown in example 1. Comprising animal and/or plant-based proteins in an addition rate of 4-20 grams. CHO contributes to 20-50 gram and 3-12 grams fat comprising preferably unsaturated fatty acids >20% and <20% saturated fatty acids. The micronutrients at least comprising of vitamin D (0.5-2.5 microgram) and magnesium (10-40 mg). Optionally flavors, colorants etc. are added.

### Example 5

A nutritionally complete medical liquid food composition was prepared to be enterally consumed, more preferably via tube nutrition by subjects >36 months of age. Per 100mL, the composition comprises of 1.2-2.5 gram fiber comprising arabinoxylan, beta-glucan, pectin and resistant starch. Digestible carbohydrates 11-19 gram and 3-7.5 gram intact or isolated proteins were either derived from plant or animal (mammal) sources. Fats contributed to 2.5-6 gram/100 mL.

### Example 6

A plant-based powder food composition was prepared for oral use by toddlers 12-36 months of age. The powder has the be dissolved in water prior to use. Per 100 grams, the composition comprises 1-4 En % fiber from the following sources: arabinoxylan, beta-glucan, pectin and resistant starch. Protein was derived from all but animal and mammalian sources at 6-10 En %. Digestible carbohydrate comprising 36-56 En % and plant derived fats 30-45 En % with saturated fatty acids <8 En %. The composition further comprised minerals, trace elements, and vitamins as known in the art.

### Example 7

A vegan food composition was prepared with nutrient sources containing all possible combinations but contained no animal derived macro-and micronutrients. The composition is for healthy subjects >36 months of age. The composition was a powder food composition to be dissolved in liquid and used as supplement. Per 100 gram dry weight the nutritional composition comprises of 5-15 grams fiber. 20-40 grams protein from all sources except those of animal and/or mammalian sources, 25-65 grams digestible carbohydrates and 4-20 grams fat of which <20% saturated fatty acids. Optionally flavors etc. are added.

### Example 8

A ready to eat vegan food composition excluding all animal and mammalian derived sources was prepared. The composition was in the form of for example but not limited to bars, biscuits, snacks, cereals, spoonables etc. The composition was prepared to be consumed orally as supplement to the habitual diet of subjects >36 months of age. Per 100 gram dry weight the preferred plant-based fiber sources may comprise of arabinoxylan, beta-glucan, pectin and resistant starch preferably in an addition rate of 7.5-15 grams. Protein comprising fruit, vegetable, cereal, plant sources contributing to 15-30 grams. Comprising of 30-45 grams digestible carbohydrates and 10-25 grams plant derived fat. Micronutrients were provided in quantities as known in the state in the art.

### Example 9

A spray dried fiber supplement, not nutritionally complete, composed by mixing 49% arabinoxylan, 21% beta-glucan, 18% pectin and 12% resistant starch was prepared.

1.-26. (canceled)

27. A nutritional composition comprising a fiber mixture comprising arabinoxylan, beta-glucan, pectin, and resistant starch, wherein the nutritional composition comprises between 0.4 to 5 g per 100 kcal of the fiber mixture, and wherein the fiber mixture comprises at least 75 wt % of fibers selected from the group consisting of arabinoxylan, beta-glucan, pectin, and resistant starch.

28. The nutritional composition of claim 27, wherein the fiber mixture is gluten free.

29. The nutritional composition of claim 27, wherein the arabinoxylan is rice, wheat, psyllium, or corn bran arabinoxylan, preferably corn bran arabinoxylan.

30. The nutritional composition of claim 27, wherein one or more of the fibers are hydrolysed.

31. The nutritional composition of claim 27, wherein pectin is low viscosity pectin having a degree of polymerisation of less than 6000 or a molecular weight of less than 150 kDa.

32. The nutritional composition of claim 27, wherein the fiber mixture comprises at least 80 wt % of fibers selected from the group consisting of arabinoxylan, beta-glucan, pectin, and resistant starch.

33. The nutritional composition of claim 27, further comprising inulin, fructopolysaccharides, fructooligosacccharides or mixtures thereof.

34. The nutritional composition of claim 27, wherein the fiber mixture comprises between 40-60%, arabinoxylan based on total fiber content.

35. The nutritional composition of claim 27, wherein the fiber mixture comprises between 10-30%, beta-glucan based on total fiber content.

36. The nutritional composition of claim 27, wherein the fiber mixture comprises between 10-30%, pectin based on total fiber content.

37. The nutritional composition of claim 27, wherein the fiber mixture comprises between 5-25%, resistant starch based on total fiber content.

38. The nutritional composition of claim 27, wherein the fiber mixture comprises arabinoxylan, beta-glucan, pectin, and resistant starch in a weight ratio of 4-6:1-3:1-3:0.5-2.5.

39. The nutritional composition of claim 27, wherein the beta-glucan is oat-derived beta-glucan.

40. The nutritional composition of claim 27, wherein the resistant starch is selected from high amylose corn starch, cassava starch or potato starch.

41. The nutritional composition of claim 27, wherein the nutritional composition is gluten free.

42. The nutritional composition of claim 27, wherein the composition is low in lactose.

43. A method of administering to a subject a fiber mixture comprising arabinoxylan, beta-glucan, pectin, and resistant starch, wherein the fiber mixture improves intestinal health and/or preventing impaired intestinal health in a subject in need thereof.

44. The method of claim 43, wherein the subject is a subject with coeliac disease.

45. The method of claim 43, wherein the fiber mixture is gluten free and optionally lactose free.

46. The method of claim 43, wherein improving intestinal health and/or preventing impaired intestinal health involves 1) an intestinal short chain fatty acid production to intestinal gas production ratio of at least 500 and 2) an intestinal short chain fatty acid production of at least 4 mmol/g fiber mixture.

47. The method of claim 43, wherein the fiber mixture comprises arabinoxylan, beta-glucan, pectin, and resistant starch in a weight ratio of 4-6:1-3:1-3:1.

48. The method of claim 43, wherein the fiber mixture is comprised in a nutritional composition.

49. The method of claim 43, wherein the mixture comprises arabinoxylan, beta-glucan, pectin, and resistant starch in a weight ratio of 4-6:1-3:1-3:1.
